(19) United States
(12) Patent Application Publication
(10) Pub. No.: US 2005/0037357 A1
(43) Pub. Date: Feb. 17, 2005

(54) Method for analyzing translation-controlled gene expression

(76) Inventor: Christoph Charle, Berlin (DE)

Correspondence Address: Kevin C Hooper;Bryan Cave, 1290 Avenue of the Americas, New York, NY, 10104-3300, US

(21) Appl. No.: 10/497,128

(22) PCT Filed: Nov. 25, 2002
(86) PCT No.: PCT/EP02/13214

(30) Foreign Application Priority Data
Nov. 29, 2001 (DE) 101 58 517.9

(51) Int. Cl. C12Q 1/68; C12M 1/34

(52) U.S. Cl. 435/6; 435/287.2

(57) ABSTRACT

The present invention relates to a method for analyzing gene expression which makes it possible, taking account of the translation state present in a cell type, tissue or organism, to correlate reliably the amount of mRNA transcribed from a gene to be investigated with the amount of protein translated from this mRNA. Determination of the translation efficiency of all mRNA variants which are transcribed from a gene to be investigated and which code for a particular protein makes it possible inter alia to identify the mRNA variant preferentially translated in a particular cell type, tissue or organism. On the basis of the amount and of the translation efficiency of the preferentially translated mRNA variant coding for a protein to be investigated it is possible to predict reliably the amount of the protein expressed in a cell type, tissue or organism.

The invention relates to a method in the area of transcription analysis and comprises in particular methods and kits for analyzing translationally controlled gene expression. The method is based on analysis of the translation efficiency of the 5′ UTR of mRNA variants transcribed from one or more genes to be investigated. The data on the translation efficiency of the various mRNA variants transcribed from one or more genes to be investigated are preferably part of a database system which, together with a specially designed tool for transcription analysis, enables a precise prediction to be made about amounts of protein in the cell type, tissue or organ to be investigated through identification and quantification of the various mRNA variants transcribed from one or more genes.

The products of gene expression, proteins, are carriers of cellular functions. It has been possible to show that regulation of gene expression plays an essential part in biological processes such as embryogenesis, tissue repair, aging or neoplastic transformation. Gene expression in eukaryotes is controlled at the level of transcription, post-transcriptionally (polyadenylation of the RNA, mRNA splicing, export of mature mRNA from the nucleus into the cytoplasm or targeted degradation of the RNA), the level of translation or post-translationally [0]. Control of expression at the level of translation represents a novel key regulatory mechanism for controlling gene expression [1]. Translation-controlled expression has been demonstrated for various growth factors, cytokines, hormone receptors, protein kinases, transcription factors, components of the translation apparatus and for regulators of the cell cycle and of apoptosis [2, 3, 4, 5 and 6]. The mRNAs which code for genes whose expression is under translational control are distinguished by a remarkable structure. The 5′ untranslated region (5′UTR) of most mRNAs is normally between 10 nucleotides (N) and 200 N long [7, 8]. About two thirds of mRNAs which code for protooncogenes or factors involved in cell division have 5′ UTRs which are longer than 200 N and/or comprise more than one start codon. The mechanisms known to date which, with the aid of the 5′ UTR of an mRNA, control the initiation of protein biosynthesis are described in detail below.

Regulation of translation by long, structured 5′ UTRs: stable secondary structures and sequence segments which comprise a high proportion of guanine and cytosine bases are able, when they are present in the 5′ UTR of an mRNA, to inhibit very efficiently the CAP-dependent initiation of protein biosynthesis according to the ribosome scanning model [1]. In vitro investigations have shown that a hairpin structure in the 5′ UTR of an mRNA having a free energy of 30-70 kcal./mol is able to inhibit translation effectively. Thus, it was also possible to show that mRNAs coding for a particular protein and having a 5′ UTR exhibiting such a structure are translated only very weakly, whereas mRNAs coding for the same protein and having a shorter 5′ UTR with a weaker structure are translated considerably more efficiently [5, 9].

Regulation of translation by upstream open reading frames (uORFs): The ribosome scanning model of translation initiation states that protein synthesis starts at the 5′-proximal start codon [10, 11]. A number of mRNAs with long 5′ UTRs which comprise one or more additional start codons upstream from the first start codon of the coding region, or one or more uORFs, have an inhibitory effect on translation of the downstream coding region [6]. mRNAs which code for a particular protein and whose 5′ UTR is comparatively short and comprises no additional start codons or uORFs are translated considerably more efficiently than mRNAs which code for the same protein and which a long 5′ UTR which comprises one or more additional start codons or uORFs, [1, 2, 3, 6, 12 and 13].

Regulation of translation by internal ribosome entry sites (IRES): Internal initiation of translation was originally discovered in picornaviruses whose mRNAs have no 5′ CAP structure and have a structured 5′ UTR which is approx. 1000 N long and additionally comprises a large number of uORFs. Despite the structure of the 5′ UTR, which effectively inhibits initiation of translation according to the ribosome scanning model [11], the RNA of picorna viruses is efficiently translated in vitro and in vivo. The secondary structures in the 5′ UTR of picorna virus RNA favor the binding of ribosomal subunits and CAP-independent initiation of translation (internal ribosome entry sites→IRES). 5′ UTRs with similar structures have also been discovered in the RNA of various other viruses [14, 15]. It has likewise been possible to detect one or more IRES in the 5′ UTR of various cellular mRNAs which are transcribed in eukaryotes [16, 17, 18, 19, 20, 21, 22, 23 and 24]. mRNAs whose 5′ UTR comprises an IRES can be translated in cells which overexpress the eukaryotic initiation factor eIF 4E, independently of a 5′-⁷methyl-G cap structure [6, 11]. It was also possible in this connection to demonstrate that an mRNA coding for a particular protein and having a short, weakly structured 5′ UTR is translated considerably more efficiently than an mRNA which codes for the same protein but whose 5′ UTR comprises an IRES [24]. One or more IRES elements in the 5′ UTR of an mRNA enables efficient translation of this mRNA after a viral infection or in eIF4E-overexpressing cells. Under normal conditions, structured 5′ UTRs with such a length prevent CAP-dependent initiation of translation.

It was possible to show that a plurality of mRNA variants are transcribed from genes whose expression is regulated at the level of translation. All mRNA variants transcribed from a particular gene have an identical sequence of the coding region. In most of the investigated cases, the principal transcript has a long structured 5′ UTR, whereas the subsidiary transcripts have shorter 5′ UTRs with weaker structures. The origin of these mRNA variants is attributable to the use of different transcription start sites and alternative splicing of the pre-mRNA [2, 12, 24].

For example, two mRNA variants are transcribed from the bcl-2 gene. The principal transcript of the bcl-2 gene has a 5′ UTR which is more than 1000 N long and comprises a plurality of uORFs. The subsidiary bcl-2 transcript has a 5′ UTR which is approx. 80 N long and is weakly structured, and is preferentially translated. The proportion of the subsidiary transcript is about 5% of the total amount of bcl-2 mRNA [2, 12]. Doubling of the transcription rate of the preferentially translated bcl-2 transcript, induced by external influences such as radiation, chemicals, cytostatics, hormones, cytokines, growth factors or stress, leads to a doubling of the protein concentration. The total amount of bcl-2 mRNA increases overall by 5%. It is not possible with conventional methods of transcription analysis [26, 29] to determine these changes accurately enough to be able to predict a change in the amount of protein.

Proteins such as growth factors, cytokines, hormone receptors, protein kinases, transcription factors, components of the translation apparatus and regulators of the cell cycle and of apoptosis play a crucial part in the development and pathogenesis of neuro-degenerative disorders, autoimmune diseases or cancer. The development of multi-drug resistant tumor cells and some regions of the so-called escape from immunity are likewise influenced by the abovementioned proteins [25]. Expression of many of the genes which code for these proteins is regulated at the level of translation [1, 6]. The change in the amount of these proteins can be analyzed with the aid of the methods summarized by the term “proteomics” [30]. However, all known methods for analyzing and/or quantifying proteins are subject to restrictions which, for example, include the limited resolving power of 2D gels, the selectivity of methods for staining proteins or the availability of antibodies. In addition, almost all methods for analyzing proteins are time-consuming, laborious and, in some cases, associated with considerable apparatus costs, so that they cannot be employed straight-forwardly in clinical routine or high-throughput procedures.

In order to avoid the problems associated with proteomics, generally the change in the amount of a particular protein is predicted with the aid of the change in the amount of mRNA which codes for this protein. The methods with whose aid it is possible to determine the mRNA amount which is transcribed from one or more genes include Northern blotting, slot and dot blotting, nuclease protection assays, PCR and DNA arrays [26]. Especially the PCR-based methods and DNA arrays for transcription analysis make it possible to analyze large amounts of samples, because their manipulation is relatively uncomplicated and can be automated. In current laboratory practice, the amount of mRNA transcribed from a gene is determined by detecting the coding region of this mRNA. It has been possible to show that the amount of mRNA coding for a particular protein is not a sufficiently accurate indicator of the amount of the corresponding protein actually present, because in more than 50% of investigated genes the detected amount of protein does not correlate with the detected amount of RNA [29]. If the expression of a particular gene is regulated at the level of translation, it is possible with the methods detailed above to determine only the total of all the mRNA variants transcribed from this gene.

A relatively precise estimate of the amount of protein present in a tissue or cell type can be achieved by analyzing the transcripts bound to polysomes, because they represent the actively translated mRNA [29, 31, 32 and 33]. The number of polysome-bound mRNA molecules is a reliable indicator of the translation rate of the corresponding proteins, because it is generally accepted that control of translation takes place mainly during the initiation phase [29, 34]. The isolation of polysome-bound mRNA requires isolation of cytoplasmic RNA under conditions which prevent dissociation of RNA-protein complexes or RNA-ribosome complexes. Polysomes are then separated from monosomes and unbound mRNA by ultracentrifugation through a sucrose gradient [29, 31, 32 and 33]. Separation of nuclear RNA and cytoplasmic RNA [36] and the subsequent ultracentrifugation step make it difficult to automate the method and thus process a large number of samples in parallel.

In areas such as clinical diagnosis or industrial drug research, which depend on automated methods in order to ensure high sample throughput, there is a need for methods for carrying out advantageous expression analyses which make reliable prediction of the expressed amount of protein possible. A precise prediction of amounts of protein by transcription analyses makes it possible to describe functional connections in cells, tissues or organisms which make it possible to determine effects, side effects and target molecules of drugs. However, the prior art expression analysis methods which can be integrated into automated systems or high throughput routines take no account of the translational state of the cell, because mRNAs coding for one or more proteins to be investigated are detected only by means of their coding region. These systems therefore do not allow any reliable prediction to be made about amounts of protein or description of functional connections in the cells, tissues or organisms to be investigated. Although expression analysis methods which take account of the translational state of the cells, tissues or organisms to be investigated, such as, for example, comparative analysis of polysomal and non-polysomal RNA, permit a reliable prediction to be made about amounts of protein, they are unsuitable because of their laborious nature for employment in high throughput procedures or in routine clinical diagnosis.

One object of the present invention is to provide an advantageous method for expression analysis of a gene.

The present invention relates to a method for analyzing gene expression, which method enables, while taking into account the translational state present in a cell type, tissue or organism, a reliable correlation to be made between the amount of mRNA transcribed from a gene to be investigated and the amount of protein translated from this mRNA. Determination of the translation efficiency of all mRNA variants transcribed from a gene to be investigated and coding for a particular protein makes it possible inter alia to identify the mRNA variant preferentially translated in a particular cell type, tissue or organism. It is possible on the basis of the amount and of the translation efficiency of the preferentially translated mRNA variant coding for a protein to be investigated to make a reliable prediction of the amount of the protein expressed in a cell type, tissue or organism. The method enables simultaneous analysis of the translationally controlled expression of a multiplicity of genes and thus analysis of functional connections in a cell type, tissue or organism. The method makes it possible to predict the amount of one or more proteins to be investigated in a tissue or cell type through determination of the transcription rate of the preferentially translated mRNA.

The invention therefore relates to a method for analyzing the expression of at least one gene coding for a protein in a sample, which comprises ascertaining where appropriate the number and identity of various mRNA variants of the gene to be analyzed which are present in the sample; ascertaining the respective amounts of the various mRNA variants of the gene to be analyzed which are present in the sample; and ascertaining on the basis of the ascertained amounts and of the respective translation efficiency of the various mRNA variants the amount, present in the sample, of protein encoded by the gene to be analyzed.

The sample is usually a composition which includes cells, a tissue or parts of an organ. It can be for example a biopsy or cells in cell culture. The sample is preferably derived from a culture of mammalian cells, from a tissue or an organ of a mammal.

The sample is usually not directly analyzed itself; on the contrary, from it a composition which comprises nucleic acid which is mRNA or is derived therefrom is obtained or prepared. This composition is preferably a preparation, obtained or prepared from the sample, of total RNA or polyA+ RNA. The nucleic acid present in the composition may likewise be cRNA or cDNA. Preparations of these types can be prepared simply from a composition comprising mRNA. The composition is analyzed and, from the values for the number, identity and/or amount of the various nucleic acid variants of the genes to be analyzed in the composition, it is possible to conclude the number, identity and/or amount of the various nucleic acid variants of the gene to be analyzed in the sample.

There is preferably initial provision of a solid matrix on which, at various points on the matrix, at least two different single-stranded nucleic acids are immobilized (=probes). These probes preferably each comprise from 10 to 40 consecutive nucleotides or consist of from 10 to 40 consecutive nucleotides, each of which are part of the nucleotide sequence of the gene to be analyzed, with a first probe being complementary to part of the nucleotide sequence of a first mRNA variant or of a cDNA, corresponding to this variant, of the gene, but this first probe not being complementary to part of the nucleotide sequence of a second mRNA variant or of a cDNA, corresponding to this variant, of the gene. In addition, a second probe is complementary to part of the nucleotide sequence of the first mRNA variant or of a cDNA, corresponding to this variant, of the gene, and this second probe is likewise complementary to part of the nucleotide sequence of the second mRNA variant or of a cDNA, corresponding to this variant, of the gene. This means that the first probe is specific for the first mRNA or cDNA variant, but the second probe is able to hybridize with the first and the second mRNA or cDNA variant.

In a further step, the solid matrix can be brought into contact with the composition which has been obtained or prepared from the sample, in which case hybridization of nucleic acid molecules in the composition with one or more probes can take place. In a further step, where appropriate then the number and identity of the various variants, which are present in the composition, of nucleic acids which are encoded by the gene to be analyzed are ascertained. Likewise, the respective amounts of the various variants, present in the composition, of nucleic acids which are encoded by the gene to be analyzed are ascertained. In a final step, on the basis of the amounts ascertained in this way and of the respective translation efficiency of the various mRNA variants of the gene to be analyzed, the amount, present in the sample from which the composition was obtained or prepared, of protein which is encoded by the gene is ascertained.

The solid matrix may also comprise a third probe which is able to hybridize with a third mRNA variant or the corresponding cDNA, because it is complementary thereto. It may also, owing to complementarity, hybridize with the first and the second mRNA variant or the corresponding cDNA. The third mRNA variant or the corresponding cDNA is, however, not recognized by the first and the second probe. The probes are thus defined so that the first mRNA variant is recognized by all three probes, the second mRNA variant only by the second and the third probe, and the third mRNA variant only by the third probe. The number of probes necessary to differentiate more different mRNA variants is correspondingly higher. It is clear to the skilled worker that more probes than theoretically necessary to differentiate the various mRNA variants can be employed.

Normally, at least one of the probes immobilized on the solid matrix comprises a nucleotide sequence which is part of the coding region of the gene to be analyzed. The probes immobilized on the matrix may in various embodiments “cover” the complete genomic nucleotide sequence of the 3′ noncoding region, of the 5′ noncoding region or of the complete noncoding region of the gene to be analyzed. Finally, the probes may also encompass the complete genomic nucleotide sequence of the gene to be analyzed. Moreover, the nucleotide sequences of the individual probes may overlap.

It is also preferred for one or more probes each of which comprise parts of the nucleotide sequence of bacterial genes, plant genes and/or housekeeping genes of the organism from which the sample originates to be immobilized on the solid matrix. These probes normally have a length of from 10 to 40 nucleotides. Examples of housekeeping genes are, for example, genes which code for β-actin, GAPDH or L32.

It is particularly preferred for the solid matrix to be configured as DNA array on which the probes are immobilized in the form of spots.

2 different mRNA variants may be transcribed from the genes to be analyzed; however, it is also possible for 3 or more different variants to be transcribed. The different variants may also differ at the 5′ end and/or at the 3′ end and/or represent different splice forms of the gene.

The invention also relates to a solid matrix as described for the method of the invention.

A further aspect of the invention is a kit for expression analysis of at least one gene in a sample. The kit comprises as component 1 a solid matrix as previously described, and as component 2 a storage medium on which the respective translation efficiencies of the various mRNA variants of the gene to be analyzed are stored. It may additionally include a device for determining the respective amounts of nucleic acid which are bound to the respective probes after a nucleic acid-containing composition has been brought into contact with the solid matrix. Preferred embodiments of the solid matrix of component 1 correspond to preferred embodiments of the matrix in the described method. Further transcription profiles may be present in component 2. The transcription profiles in this connection may be first derived from cells, tissues or organisms altered by a disease. Examples of such diseases are cancer, neurodegenerative disorders, autoimmune diseases, chronic disorders of the elderly, cardiovascular disorders, viral diseases and drug resistances.

The transcription profiles may be in particular derived from tumor cells which have been treated with one or more therapeutic agents. The further transcription profiles in component 2 may be stored on the same storage medium as the translation efficiencies, but they may also be stored on one or more separate storage media.

A further aspect of the invention is the use of the described solid matrix for determining the protein concentration in a sample, for determining or analyzing disorders, for determining or analyzing the effects of external influences on the cells to be investigated or for determining the secondary structure of RNA molecules.

The system for carrying out the method normally consists of two components. Component 1 is usually a DNA array for identifying and quantifying all mRNA variants transcribed from one or more genes to be investigated. Besides quantitative determination of the transcription of various genes, alternatively utilized transcription starting points of these genes and splice variants in the 5′ UTR and in the 3′ UTR of the mRNA variants transcribed from these genes are analyzed and quantitatively determined with the aid of the specifically designed DNA array contained in component 1. The information from a combination of nuclease protection assays, Northern blotting and quantitative RT-PCR [26] can be made possible by the DNA array. Component 2 may be a software package consisting of a database module and an analysis module. In the database, where appropriate on a storage medium, values on the translation efficiency of all the mRNA variants transcribed from the genes to be investigated under various conditions are organized. The database contains all the necessary data for a reliable prediction of the amount of a protein translated in a particular cell type, tissue or organism to be possible on the basis of a transcription profile. The analysis module embedded in component 2 ascertains, on the basis of the transcription pattern produced with component 1 and the database, the amount of the preferentially translated mRNA variant or mRNA variants which are transcribed from one or more genes to be investigated in the cell type, tissue or organism under particular conditions.

In one embodiment, the system relates to methods for determining and analyzing the effects and secondary effects of various external influences on cell types, tissues or organisms to be investigated. These external influences may include inter alia drugs (pharmaceuticals), cytokines, hormones, growth factors, environmental influences (temperature, atmospheric pressure, chemicals) or the nutrient supply. Poly A⁺ mRNA, total cellular RNA or cDNA prepared from these RNA populations from cells, tissues or organisms exposed to one or more of the abovementioned influences are analyzed with component 1. These transcription profiles are compared with transcription profiles of identical or similar cells, tissues or organisms not exposed to the abovementioned external influences. The system can be employed in drug research in order, for example in the development of novel tumor therapeutic agents, to analyze the effect on cells and the potential for the development of a multi-drug resistant phenotype.

The system may additionally comprise methods for analyzing pathological states which include inter alia neurodegenerative syndromes, cancer, autoimmune diseases, chronic disorders of the elderly, cardiovascular disorders, viral diseases and/or drug resistances. In the area of diagnosis of neoplastic diseases, the system is intended to be employed for the analysis and assessment of the potential for metastasis and the aggressiveness of a tumor, and for the analysis and assessment of the multi-drug resistance of tumors, in order to achieve an improvement in therapeutic efficiency and in order to make it possible to design individual types of therapy. The database module of component 2 is in this case extended by data records which includes transcription profiles of tumor cells produced with component 1, and clinical data on the tumor cells. In addition, these data records comprise transcription profiles, produced with component 1, of cultivated tumor cells which have been treated with various tumor therapeutic agents and data (e.g. division rate, apoptosis rate and others) on the response of these cells to the therapeutic agents (response profiles).

In a further embodiment, the invention relates to methods for ascertaining the secondary structure of mRNA molecules. It is possible in particular to ascertain reliably the secondary structure of RNAs with catalytic activity, called ribozymes, or regulatory regions of mRNAs such as, for example, internal ribosome entry sites (IRES). The specific design of the DNA array (component 1) represents a complete replacement for the nuclease protection assay [26] used in common laboratory practice. It is not always possible in conventional nuclease protection assays to ascertain unambiguously which region of the probe target duplex is double-stranded, i.e. protected from nucleases. A considerable advantage of the present invention is that the exact sequence of the “protected” regions is indicated. The RNA molecules to be investigated are subjected to a partial RNAse digestion and subsequently hybridized with the DNA arrays. The DNA array (component 1) makes it possible to identify double-stranded regions in an RNA molecule to be investigated. In conjunction with common algorithms for calculating secondary structures of nucleic acids [24], these data can be used to produce a reliable model of the folding of the RNA molecule to be investigated. The production of three-dimensional models of IRES elements, enzymatically active RNAs (ribozymes) or other RNA structures without the use of spectroscopic methods or of X-ray structural analysis is thus made possible for the first time.

### DESCRIPTION OF PREFERRED EMBODIMENTS OF THE INVENTION

Component 1 (DNA Array)

Component 1 is preferably a DNA array which is specifically adapted and designed for the requirements of the system and with whose aid it is possible to identify and quantify in an amount of sample nucleic acids, which may be total RNA, polyA⁺ mRNA or cDNA, that mRNA variant which is transcribed from one or more genes to be investigated. The DNA array will comprise probe nucleic acids for detecting all mRNA variants necessary for the analysis, diagnosis and interpretation of the effect of one or more particular external influences on a cell type, tissue or organism to be investigated. These external influences may include inter alia a change in the oxygen partial pressure, in the nutrient supply, in the temperature, in the atmospheric pressure, and the effect of cytokines, hormones, cytostatics or other drugs, and pathological changes such as cancer, neurodegenerative syndromes, autoimmune diseases, cardiovascular disorders, viral infections and drug resistances.

Design and Interpretation of the DNA Array

In one embodiment, a single-stranded nucleic acid, which may be DNA, RNA or a nucleic acid analogue such as PNA (peptide nucleic acid) [27], and whose base sequence is identical to the base sequence of the 5′ noncoding region (5′ NCR), of the coding region (CR) and, where appropriate, of the 3′ noncoding region (3′ NCR) of the gene to be investigated, is divided into oligonucleotides with a length Lₓ of at least 10 and at most 40 nucleotides. The equilibrium melting temperature of all the oligonucleotides should be the same (Tₘ=const.). The exact length Lₓ of the individual oligonucleotides is a function of the given equilibrium melting temperature (Tₘ) and their base composition (% GC), i.e. L_{x(Tm=const)}=f_{(Tm:% GC)}[37, 38, 39, 40, 41 and 42] and is
L_{x(Tm=const.)}=10+nnucleotides.

The resolving power of the method depends on the length Lₓ of the segments, referred to hereinafter as probe nucleic acids. The length Lₓ of the probe nucleic acids, and thus the resolving power along the sequence to be investigated, varies depending on the content of GC nucleotides within the sequence to be investigated. The resolving power A of the method can be increased by overlapping segments which are immobilized in parallel to a first set of segments on a solid matrix, apart from one nucleotide (A=Lₓ/n, where n=1→Lₓ).

The synthetic oligonucleotides corresponding to the base sequence of the gene to be investigated (referred to hereinafter as probe nucleic acids) are bound to a solid matrix, preferably covalently. This solid matrix may be a flat area (DNA array), a fiber or the surface of a microparticle [45] consisting of plastic (e.g. polypropylene, nylon), polyacrylamide, nitrocellulose or glass. The covalent linkage of the oligonucleotide probes to the solid matrix may take place on the one hand by in situ oligonucleotide synthesis [46, 47, 48 and 49] or application of modified oligonucleotides, which may be DNA, RNA or PNA, to an activated surface [50, 51]. Equipment for printing DNA arrays is produced and marketed by a number of suppliers [57]. The probe nucleic acids are synthesized by standard biotechnology laboratory protocols [52]. The covalently bonded probe nucleic acids are arranged in a sequence corresponding to the base sequence of the gene to be investigated, so that a DNA strand which harbors the base sequence of the gene to be investigated is simulated (remodeled) in the 5′-3′ direction on the matrix (“tiled array”). The probe nucleic acids immobilized on the matrix in this way are divided into three region.

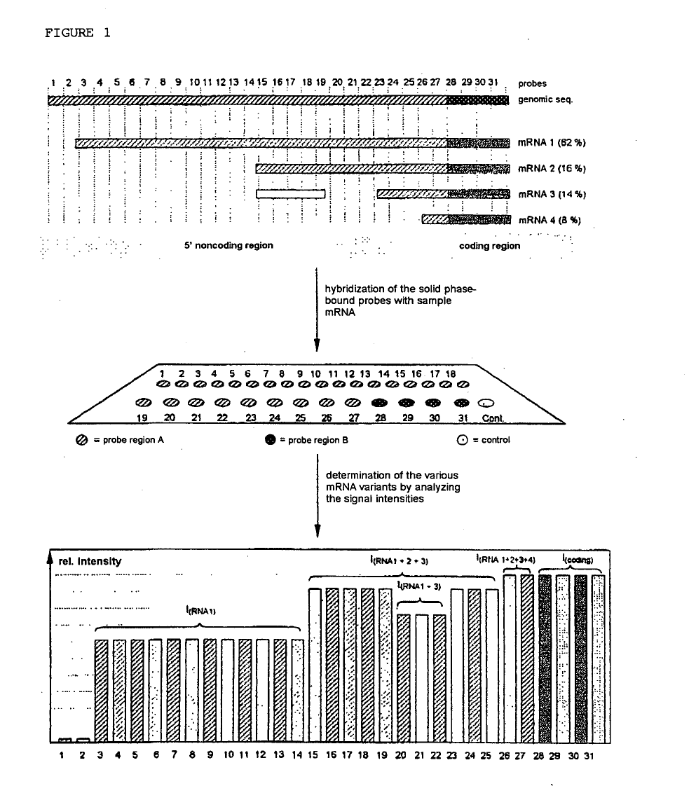

FIG. 1 shows a diagrammatic depiction of the probes of various mRNA variants transcribed from a gene to be investigated, the solid phase-bound probes (array) and an analysis of the hybridization data.

Region A, region B or region C contains all probe nucleic acids whose base sequence is identical to the base sequence of the 5′ noncoding region (5′ NCR), of the coding region (CR) or of the 3′ noncoding region (3′ NCR) of the gene to be investigated.

The matrix-bound probe nucleic acids are brought into contact with single-stranded sample nucleic acid, which may be mRNA, cRNA or cDNA [26], under conditions which allow duplex formation by hybridization of complementary single-stranded nucleic acids. If cDNA is employed as sample nucleic acid, the base sequence of the probe nucleic acids is identical to that of the codogenic strand (sense strand) of the gene to be investigated. If mRNA or cRNA are employed as sample nucleic acid, the base sequence of the probe nucleic acids is identical to that of the noncodogenic strand (antisense strand) of the gene to be investigated. To detect the hybridization events, either the sample nucleic acids may be radiolabeled or labeled with fluorophores or parts of a binding pair (biotin, streptavidin) [26], or the probe nucleic acids are labeled with fluorophores or parts of a binding pair (biotin, streptavidin) [27, 28].

Sequence segments of the gene to be investigated which are not present in the base sequence of the mRNA variants transcribed from this gene do not hybridize with the solid phase-bound probe nucleic acids (see FIG. 1). These sequence segments include intron sequences, sequence segments of the 5′ NCR (probe region A) of the gene to be investigated which are located upstream from the individual start of transcription of a particular mRNA variant, and sequence segments in the 3′ NCR (probe region C) of the gene to be investigated which are located downstream from the 3′ end of a particular mRNA variant. The coding region of all mRNA variants transcribed from the gene to be investigated hybridizes with the probe nucleic acids whose base sequence is identical to that of the coding region (probe region B) of the gene to be investigated.

The signal intensity of the hybridization signals detectable in probe region B (I_{B(CR)}) are equal to the total of the signal intensities of the detectable hybridization signals (Σ(I_{(RNA1)}, I_{(RNA2)}, . . . , I_{(RNAn)}) of the individual mRNA variants which are transcribed from a gene to be investigated.
I_{B(CR)}=(Σ(I_{(RNA1)}, I_{(RNA2)}, . . . , I_{(RNAn)})

Hybridization signals detectable in probe region A or probe region C (I_{A(5′-NTR)} or I_{C(3′-NTR)}) which display the same signal intensity as the hybridization signals detectable in probe region B (I_{B(CR)}) correspond to sequence motifs outside the coding region which are present in all mRNA variants transcribed from the gene to be investigated.

The transcription start which is the furthest distance upstream (in the 5′ direction) from the coding region is indicated by the first probe nucleic acid in probe region A showing a detectable hybridization signal after hybridization with sample nucleic acid (¹I_{A(1)}). If only one mRNA variant is transcribed from this transcription start, then
¹I_{A(1)}=I_{B(CR)},
with all probe nucleic acids in probe region A showing hybridization signals of the same intensity which are identical to the signal intensity of the hybridization signals in probe region B. The following applies:
¹I_{A(1)}=¹I_{A(2)}=¹I_{A(3)}. . . =¹I_{A(n)}=I_{B(CR)}.

In order to compensate for variations in the intensity of the hybridization signals in probe region A, B or C and to make it possible to estimate errors (standard deviation, deviation of the mean) of the measurements, the average or the median of the measured signal intensities is calculated:
(¹I_{A(1)}+¹I_{A(2)}+¹I_{A(3)}+. . . +¹I_{A(n)})/n=Ø¹I_{A}=I_{B(CR)}=ØI_{B(n)}=(I_{B(1)}+I_{B(2)}+I_{B(3)}+. . . +I_{B(n)})/n

If additional mRNA variants are transcribed from starting points located downstream from the first transcription start, the intensity of the hybridization signals of the mRNA variant 1 transcribed from the first transcription start in probe region A is less than the signal intensities of the hybridization signals in probe region B. The following applies:
¹I_{A(1)}=¹I_{A(2)}=¹I_{A(3)}=. . . =¹I_{A(n)}<I_{B(CR)}, or
(¹I_{A(1)}+¹I_{A(2)}+¹I_{A(3)}+. . . +¹I_{A(n)})/n=Ø¹I_{A}<I_{B(CR)}=ØI_{B(n)}

The position of the next (transcription start 2) transcription start located downstream from the first transcription start (transcription start 1) is indicated by the first probe nucleic acid in probe region A (²I_{A(1)}), which shows a hybridization signal of higher intensity after hybridization with sample nucleic acid than the probes which hybridize specifically with the mRNA variant (RNA 1) transcribed from transcription start 1. If two mRNA variants are transcribed from a gene to be investigated from different transcription starts, i.e. with 5′ UTRs of different lengths, then:
²I_{A(1)}=I_{B(CR)},
in which case all probe nucleic acids in probe region A which hybridize specifically with RNA 2 show hybridization signals of the same intensity which are identical to the signal intensity of the hybridization signals in probe region B. The following applies:
²I_{A(1)}=²I_{A(2)}=²I_{A(3)}=. . . =²I_{A(n)}=I_{B(CR)}, or
(²I_{A(1)}+²I_{A(2)}+²I_{A(3)}+. . . +²I_{A(n)})/n=Ø²I_{A}=I_{B(CR)}=ØI_{B(n)}

Since the signal intensity of the hybridization signals detectable in probe region B (I_{B(CR)}) is equal to the total of the signal intensities of the detectable hybridization signals (Σ(I_{(RNA1)}, I_{(RNA2)}, . . . I_{(RNAn)}) of the individual mRNA variants transcribed from a gene to be investigated, then:
I_{B(CR)}=Σ(I_{(RNA1)}, I_{(RNA2)}, . . . , I_{(RNAn)})

Based on the hybridization signals in probe region A and B, this results in:
I_{B(CR)}=ØI_{B(n)}=Ø²I_{A}=Σ(I_{(RNA1)}, I_{(RNA2)}), where
I_{(RNA1)}=(¹I_{A(1)}+¹I_{A(2)}+I_{A(3)}+. . . +¹I_{A(n)})/n=Ø¹I_{A} and
I_{(RNA2)}=[(²I_{A(1)}+²I_{A(2)}+²I_{A(3)}+. . . +²I_{A(n)})−(¹I_{A(1)}+¹I_{A(2)}+¹I_{A(3)}+. . . +¹I_{A(n)})]/n=Ø²I_{A}−Ø¹I_{A}

If n mRNA variants are transcribed from a gene to be investigated from n−1 starting points which are located downstream from a first transcription start, the intensity of the hybridization signals of the mRNA variants transcribed from all starting points apart from the last before the first start codon of the coding region in probe region A is less than the signal intensities of the hybridization signals in probe region B (see above). The following applies:
Ø¹I_{A}, Ø²I_{A}, Ø³I_{A}, . . . , Ø⁽ⁿ⁻¹⁾I_{A}<ØⁿI_{A}=I_{B(CR)}=ØI_{B(n)}=Σ(I_{(RNA1)}, I_{(RNA2)}, I_{(RNA3)}, . . . , I_{(RNAn)})
where
I_{(RNA1)}=(¹I_{A(1)}+¹I_{A(2)}+¹I_{A(3)}+. . . +¹I_{A(n)})/n=Ø¹I_{A}
I_{(RNA2)}=[(²I_{A(1)}+²I_{A(2)}+²I_{A(3)}+. . . +²I_{A(n)})−(¹I_{A(1)}+¹I_{A(2)}+¹I_{A(3)}+. . . +¹I_{A(n)})]/n=Ø²I_{A}−Ø¹I_{A}
I_{(RNA3)}=[(³I_{A(1)}+³I_{A(2)}+³I_{A(3)}+. . . +³I_{A(n)})−(²I_{A(1)}+²I_{A(2)}+²I_{A(3)}+. . . +²I_{A(n)})]/n=Ø³I_{A}−Ø²I_{A}
I_{(RNAn)}=[(ⁿI_{A(1)}+ⁿI_{A(2)}+ⁿI_{A(3)}+. . . +ⁿI_{A(n)})−(ⁿ⁻¹I_{A(1)}+ⁿ⁻¹I_{A(2)}+ⁿ⁻¹I_{A(3)}+. . . +ⁿ⁻¹I_{A(n)})]/n=ØⁿI_{A}−Øⁿ⁻¹I_{A}

The proportion of each mRNA variant in the total amount of the various mRNA variants transcribed from a gene to be investigated can be determined on the basis of the hybridization intensities.

If two mRNA variants arising through alternative splicing of the pre-mRNA are transcribed from a gene to be investigated from one transcription starting point, the transcription start of the two mRNA variants is indicated by the first probe nucleic acid in probe region A, which shows a detectable hybridization signal after hybridization with sample nucleic acid (¹I_{A(1)}). The intensity of the hybridization signals corresponds to the total of the intensities of the two mRNA variants (spliced: mRNAs and unspliced: mRNA) and is equal to the intensity of the hybridization signals in probe region B
¹I_{A(1)}=(¹I_{A(1)}+¹I_{A(2)}+¹I_{A(3)}+. . . +¹I_{A(n)})/n=Ø¹I_{A}=I_{B(CR)}=Σ(I_{(RNAS)}, I_{(RNA)}

In the region of the splice site, the intensity of the hybridization signals (^{1s}I_{A(1)}) is lower than the
(^{1s}I_{A(1)}+^{1s}I_{A(2)}+^{1s}I_{A(3)}+. . . +^{1s}I_{A(n)})/n=Ø^{1s}I_{A}<Ø¹I_{A}=I_{B(CR)}=Σ(I_{(RNAS)}, I_{(RNA)})
I_{(RNAS)}=(^{1s}I_{A(1)}+^{1s}I_{A(2)}+^{1s}I_{A(3)}+. . . +^{1s}I_{A(n)})/n=Ø¹I_{A}
I_{(RNA)}=[(¹I_{A(1)}+¹I_{A(2)}+^{1s}I_{A(3)}+. . . +¹I_{A(n)})−(^{1s}I_{A(1)}+^{1s}I_{A(2)}+^{1s}I_{A(3)}+. . . +^{1s}I_{A(n)})]/n=Ø¹I_{A}−Ø^{1s}I_{A}

Whether it is necessary to represent/remodel the entire genomic sequence to be investigated by probe nucleic acids in probe regions A and C, or only the sequence regions which flank the transcription starts and splice sites, depends on the area of use of component 1. If the expression of known mRNA variants transcribed from one or more genes to be investigated is to be measured, only the number of probe nucleic acids necessary for identifying and quantifying the individual mRNA variants needs to be immobilized in probe region A or C. If it is intended with the aid of component 1 to identify new mRNA variants or elucidate the secondary structure of an mRNA, it is necessary for probe region A and C to represent the entire genomic sequence to be investigated. The DNA array may also comprise a further region comprising probe nucleic acids which hybridize specifically with a number of mRNAs of housekeeping genes and with a selection of plasmids, bacterial or plant RNAs. This probe region serves firstly to standardize the hybridization signals in probe region A, B and C and for checking the stringency of the hybridization.

Hybridization of the DNA Array

In a further embodiment, labeled cDNA is synthesized from total RNA or polyA⁺ mRNA by reverse transcription using oligo-dT or p(dN)₆ as starter oligonucleotide. Enzymatic synthesis of cDNA by reverse transcriptase is a standard biotechnology in laboratory procedure [26]. Reverse transcription of sample RNA is carried out in the presence of dNTPs which are conjugated to a detectable group, preferably a fluorophore or a part of a binding pair. A further possibility is to convert isolated mRNA by reverse transcription into double-stranded cDNA and to synthesize labeled cRNA from the latter by in vitro transcription in the presence of rNTPs which are conjugated to detectable groups [26, 53].

In a further preferred embodiment, the probes immobilized on the DNA array are labeled. This labeling may be one or more fluorophores or part of a binding pair. After hybridization of the array with unlabeled total RNA, polyA⁺ mRNA, cRNA or cDNA and the subsequent washing steps, unhybridized (single-stranded) probe nucleic acids are removed enzymatically from the array, and the amount of probe nucleic acids remaining on the array is measured [28].

A number of fluorophores can be employed for the fluorescence labeling of the sample and probe nucleic acids, such as, for example, fluorescein, lissamine, phycoerythrin, rhodamine (Perkin Elmer Cetus), Cy2, Cy3, Cy3.5, Cy5, Cy5.5, Cy7, FluorX (Amersham) [53]. Besides the fluorophores listed here, it is also possible to employ for the labeling other fluorophores not listed here. These include all fluorophores which can be covalently linked to nucleic acids and whose excitation and emission maxima are in the infrared region, in the visible region or in the UV region of the spectrum. If sample or probe nucleic acids are labeled with parts of a binding pair such as biotin or digoxigenin, after hybridization the second part of the binding pair (streptavidin or anti-digoxigenin Ab) conjugated to a detectable label is incubated with the hybrids. The detectable label of the second part of the binding pair may be a fluorophore or an enzyme (alkaline phosphatase, horseradish peroxidase inter alia) which converts a substrate with emission of light (chemiluminescence or chemifluorescence) [54, 55].

The hybridization and washing conditions are adjusted so that the sample nucleic acids bind specifically to a particular probe nucleic acid immobilized on a solid matrix, or is able to hybridize specifically with this probe nucleic acid. This means that the sample nucleic acid binds, hybridizes or forms a duplex with an immobilized probe nucleic acid which has a sequence complementary to the sample nucleic acid, and not to an immobilized probe nucleic acid which has a non-complementary base sequence. A polynucleotide sequence is in this connection referred to as complementary to another one if the hybrid of two polynucleotides, of which the shorter (the probe nucleic acid) is a maximum of 25 N long, shows no base mismatches according to the standard rules for base pairing over the entire length of the shorter polynucleotide. In addition, a hybrid of two polynucleotides in which the shorter of the two polynucleotides is longer than 25 N must not contain more than 5% of mismatches according to the standard rules for base pairing. It is preferred for the polynucleotides to be perfectly complementary to one another; the hybrid contains no mismatches. The optimal hybridization conditions depend firstly on the length and type of probes (DNA, RNA, PNA) immobilized on a solid matrix and on the type of sample nucleic acids (DNA or RNA) employed. Generally valid parameters for specific (i.e. stringent) hybridization are described in customary handbooks and protocols for hybridizing nucleic acids [26, 56].

Signal Detection

If probe and sample nucleic acids labeled with fluorophores are employed for detecting hybridization events on the DNA array from component 1, the fluorescence emission can be measured at each sample point (spot) preferably by confocal laser scanning microscopy. Detection of hybridization events in nucleic acids by chemoluminescence or chemofluorescence can be carried out by using suitable filters and detectors likewise with equipment functioning according to the principle of the confocal laser scanning microscope. Equipment for signal detection on biochips is developed and marketed by a number of manufacturers [57].

Component 2 (Database & Analysis)

Component 2 of the system preferably consists of a database module and an analysis module. The database comprises data on the translation efficiency of all the mRNA variants transcribed from genes whose expression is regulated at the level of translation. The data organized in the database module describe, for example, the influence of the 5′ UTR, the influence of the coding region and of the 3′ UTR, and the influence of the cell type, tissue or organism on the translation efficiency of various mRNA variants transcribed from one or more genes. Further data records can describe the effect of external influences on the translation efficiency of the mRNA variants to be investigated. These external influences may include inter alia a change in the oxygen partial pressure, in the nutrient supply, in the temperature, in the atmospheric pressure, and the effect of cytokines, hormones, cytostatics or other drugs. The mRNA variant which is transcribed from genes with translationally controlled expression and is preferentially translated in various cell types, tissues or organisms is likewise a component of the database module of component 2.

Data Acquisition

Identification of Genes with Translationally Controlled Expression

A plurality of mRNA variants with identical coding regions but differing in the length and base sequence of their 5′ UTRs and/or 3′ UTRs are transcribed from genes with translationally controlled expression. In order to be able to determine the respective amount of the different mRNA variants transcribed from one gene, and the mRNA variant which is preferentially translated in a cell type, tissue or organism, it is necessary to know the transcription starting points, splice variants, the number of mRNA variants transcribed from a gene to be investigated, the amount of the mRNA variants transcribed in a cell type, tissue or organism, and the translation efficiency of the individual mRNA variants.

The transcription starts utilized in a cell type, tissue or organism in the 5′ noncoding region of a gene to be investigated are identified and located with the aid of nuclease protection assays, PCR methods or hybridization with DNA arrays (component 1) [26]. The mapping of splice sites, i.e. intron-exon junctions in the region of the 5′ UTR or of the 3′ UTR of the mRNA to be investigated, takes place by nuclease protection assays, PCR methods or hybridization with DNA arrays (component 1) [26]. The base sequence of the hybridization probes employed in a nuclease protection assay for investigating the transcription start(s) and the splice variants of a gene to be investigated corresponds to the base sequence of the gene to be investigated. Total cellular RNA [26, 36] or polyA⁺ mRNA [26, 43] is isolated from cell types, tissues or organisms to be investigated and is hybridized with the labeled probes, which may be cDNA or cRNA. Digestion of single-stranded regions in the hybrids, and gel electrophoretic fractionation of the resulting fragments [26, 44] take place by standard biotechnology laboratory protocols. For the mapping of the transcription starts and splice sites in the 5′ noncoding region or in the 3′ noncoding region of the gene to be investigated by PCR methods (RT-PCR), total cellular RNA [26, 36] or polyA⁺ mRNA [26, 43] is isolated from cell types, tissues or organisms to be investigated and is transcribed into cDNA by reverse transcription [26]. The PCR primers are oligodeoxy-nucleotides with which there is specific amplification of fragments which represent the 5′ portion of the coding region and the 5′ noncoding region of the gene to be investigated, and the coding region and the 5′ UTR of the mRNA variants transcribed from this gene. If the 3′ UTR of the mRNA variants transcribed from the gene to be investigated is to be mapped, the PCR primers employed are those with which it is possible to amplify fragments which represent the 3′ portion of the coding region and the 3′ noncoding region of the gene to be investigated or of the mRNA variants transcribed from this gene. To determine mRNA variants which differ in the base sequence of the 5′ UTR, one or more 3′ primers (3′ primers bind to the 3′ end of the DNA fragment to be amplified) are placed in the 5′ region of the coding region of the gene to be investigated. The population of 5′ primers (5′ primers bind to the 5′ end of the DNA fragment to be amplified) extends from the start of the coding region to beyond the first transcription starting point of the gene to be investigated. The positions and sequence of the 5′ primers are chosen so that, together with a 3′ primer, in each case there is amplification of fragments whose length increases from the first primer pair in the coding region onwards, always by 30-60 bp in each case. In order to be able to use a 3′ primer to map the transcription starting points and any splice sites present in the 5′ region of a gene to be investigated over a 2000 bp region, between 35 and 70 corresponding 5′ primers are required, depending on the resolution. The reactions are carried out with genomic DNA or plasmids which comprise the necessary regions of the gene, and mRNA from cells, tissues or organisms to be investigated. It is possible to identify transcription starting points and splice sites by comparing the fragment size and amount.

Quantitative Determination of the mRNA Variants Transcribed from a Gene to be Investigated

The proportion of each individual mRNA variant in the total amount of mRNA variants transcribed from a gene to be investigated is determined by quantitative PCR methods (TaqMan® or molecular beacons [58, 59]), multiprobe nuclease protection assays, or DNA arrays (component 1) [26]. The PCR primers correspond to those employed for identifying the mRNA variants transcribed from one or more genes to be investigated. Employed for the quantitative determination are TaqMan® probes or molecular beacons [58, 59], with which the various mRNA variants are specifically detected and quantified by means of their respective 5′ UTRs. Additionally employed are PCR primers and TaqMan® probes or molecular beacons, with which a selection of housekeeping genes is specifically detected. The template employed is cDNA synthesized by reverse transcription from total cellular RNA [26, 36] or polyA⁺ mRNA [26]. The hybridization probes, which may be cDNA or cRNA, employed in a multiprobe nuclease protection assay have different lengths, so that they can be easily distinguished from one another satisfactorily by polyacrylamide gel electrophoresis [26]. The nucleotide sequence of the hybridization probes is complementary to a nucleotide sequence of the 5′ UTR of the various mRNA variants to be investigated, and to the coding sequence of the mRNA of a selection of housekeeping genes. The quantitative PCRs and the multiprobe nuclease protection assays are carried out by standard biotechnology laboratory protocols. Preferably, the transcription rate of the mRNA variants transcribed from one or more genes to be investigated is carried out by hybridization of total cellular RNA, polyA⁺ mRNA or labeled cDNA with component 1 (DNA array) of the system (see above). The transcription rates, ascertained using the methods mentioned, of the mRNA variants transcribed from one or more of the genes to be investigated are standardized against the transcription rate of one or more housekeeping genes such as, for example, β-actin, GAPDH, L32. Quantitative determination of the mRNA variants which are transcribed in various cell types, tissues or organisms from genes with translationally controlled expression preferably takes place using the DNA array of component 1 of the system. The DNA array used hybridized with total cellular RNA, polyA⁺ mRNA or labeled cDNA isolated from cells to be investigated. The cell lines which are present in the NCI-60 panel [35] and which have been very comprehensibly characterized serve as basis here. In addition, the transcription of the mRNA variants from genes with translationally controlled expression is determined in clinical samples and other established cell lines.

Determination of the mRNA Variants Preferentially Translated in a Cell Type, Tissue or Organism

The change in the transcription rate of the mRNA variants transcribed from one or more genes, and the change in the expression rate of the corresponding proteins are normally measured as a function of various external influences. These external influences include inter alia a change in the oxygen partial pressure, in the nutrient supply, in the temperature, in the atmospheric pressure, and the effect of cytokines, hormones, cytostatics or other drugs. The change in the transcription or expression rate as a function of external influences is determined by comparing the transcription or expression rate of one or more genes to be investigated in cells, tissues or organisms which have been cultivated under ideal growth conditions with that in cells exposed to one or more of the abovementioned external influences. Total cellular RNA or polyA⁺ mRNA is isolated from cell types, tissues or organisms to be investigated. The transcription rate of the mRNA variants from one or more to be investigated is determined by qualitative RT-PCR, multiprobe nuclease protection assays or, preferably, with the aid of the DNA arrays (component 1) described above (see above). The expression rate of the corresponding genes takes place by measuring the concentration of the corresponding proteins by immunochemical methods such as Western blotting, immunoprecipitation or ELIZA [65, 66 and 67]. Since it is generally accepted that control of translation takes place mainly during the initiation phase [29, 34], the amount of protein detectable in a cell type, tissue or organism is directly proportional to the amount of the corresponding mRNA variants. The mRNA variants whose transcription rate as a function of external influences agrees with the expression rate of the corresponding protein is the mRNA preferentially translated in a particular cell type, tissue or organism. Which of the mRNA variants transcribed from a gene to be investigated is preferentially translated depends, besides the sequence of the 5′ UTR of the mRNA variants, on cell-, tissue- or organism-specifically expressed factors which influence the initiation of translation. Quantitative determination of the mRNA variants transcribed in various cell types, tissues or organisms from genes with translationally controlled expression preferably takes place using the DNA array of component 1 of the system. The DNA array is hybridized with total cellular RNA, polyA⁺ mRNA or labeled cDNA isolated from cells to be investigated. The amount of the proteins translated from the mRNA variants is determined with the aid of standard immunochemical methods. The cell lines which are present in the NCI-60 panel [35] and which have been very comprehensively characterized serve as basis here. In addition, the transcription of the mRNA variants from genes with translationally controlled expression, and the expression of these genes, is determined in clinical samples and other established cell lines.

Determination of the Translation Efficiency of the Various mRNA Variants Transcribed from the Gene to be Investigated

The rate-determining step of protein synthesis is initiation. The complexing of initiation factors, of the ribosomal subunits, and the migration of the complete ribosome to the first start codon of the open reading frame depends essentially on the length and structure, i.e. in the final analysis on the base sequence, of the 5′ UTR of the mRNA to be investigated. The translation efficiency of the various mRNA variants transcribed from one or more genes to be investigated is determined by reporter gene assays. The 5′ UTRs of the various mRNAs translated from one or more genes to be investigated are amplified with the aid of reverse transcriptase PCR [26] from total RNA or polyA⁺ mRNA or with the aid of PCR from cDNA libraries [26], and are isolated. The PCR primers are chosen so that the 5′ nucleotide of the 3′ primer corresponds to the last nucleotide of the 5′ UTR before the start codon of the coding region. The corresponding 5′ primer is located as near as possible at the transcription start of the mRNA variant to be investigated. Recognition sequences of restriction endonucleases can be integrated into the 5′ region of the PCR primers in order to facilitate ligation of the fragments into a suitable reporter gene vector (pGL3 basic inter alia; Promega) [26]. Various systems with whose aid it is possible inter alia also to determine the influence of the coding region on the translation efficiency of the mRNA variants to be investigated are employed.

Measurement of the translation efficiency in rabbit reticulocyte lysate: the various 5′ UTRs to be investigated are amplified with the aid of PCR and ligated into a plasmid vector (pGL-3/T7) between the 3′ end of the T7 promoter and the 5′ end of the gene coding for photinas pyralis luciferase [68, 69]. There are standard biotechnology laboratory protocols for the transfection and replication of plasmid vectors in suitable E. coli host strains and for the isolation of the plasmid DNA from the host organisms [26]. The plasmid vector is cut open at the 3′ end of luciferase gene with the aid of suitable restriction endonuclease.

The linearized plasmid DNA is employed as template in an in vitro transcription reaction catalyzed by a phage-encoded RNA polymerase (T7, T3 or SP6 RNA polymerase) [26]. An mRNA having a 5′ cap structure can be synthesized by adding a cap analogue [Boehringer Mannheim] to the transcription reaction in vitro. The photinas pyralis luciferase enzyme is synthesized from the in vitro synthesized photinas pyralis luciferase mRNA variants with the aid an in vitro translation system (rabbit reticulocyte lysate). Equimolar amounts of the various photinas pyralis luciferase mRNA having 5′ UTRs to be investigated are employed in the in vitro translation. The luciferase activity in the various mixtures is determined in a luminometer [26, 70]. The baseline value (100%) used for all the measurements is the luciferase activity of in vitro translation mixtures in which photinas pyralis luciferase mRNA whose 5′ UTR comprises exclusively a Kozak consensus sequence was translated [7, 8]. The influence of the various 5′ UTRs to be investigated on the translation of an mRNA in vitro is determined by these measurements. It is possible to ascertain by varying the experimental parameters whether an mRNA to be investigated can be translated independently of a 5′ cap structure, i.e. whether the 5′ UTR of this mRNA comprises an IRES element. To investigate the dependence of the translation efficiency on a 5′ cap structure, the translation efficiency of an mRNA which has a particular 5′ UTR and a 5′ cap structure is compared with the translation efficiency of an mRNA which has the same 5′ UTR but no 5′ cap structure. In order to identify a possible IRES element in the 5′ region of an mRNA to be investigated, a DNA fragment able to form a stable hairpin loop is ligated into the abovementioned reporter gene vectors between the 3′ end of the T7 promotor and the 5′ end of the 5′ UTR to be investigated. When this plasmid DNA is employed as template in an in vitro transcription reaction, the synthesized mRNA has a stable hairpin structure at the 5′ end. This structure very efficiently prevents initiation of translation according to the ribosome scanning model [1]. The ratio of the translation efficiency of mRNAs which have a particular 5 UTR and 5′ hairpin structure to the translation efficiency of mRNAs which have this 5′ UTR but no 5′ hairpin structure is formed. If this ratio is greater than 1, the translation of this mRNA can be initiated by internal ribosome entry. Ascertaining the translation efficiency of particular mRNAs by in vitro translation and subsequent determination of a reporter gene provides the basic data on the translation efficiency of one or more mRNA variants to be investigated. In this measurement system, no account is taken of the specific influence of various cell types, tissues or organisms on the translation efficiency of mRNAs to be investigated.

Measurement of the translation efficiency in vivo: in order to investigate the influence of cellular factors on the translation efficiency of one or more mRNAs to be investigated as a function of the cell type, tissue or organism, eukaryotic expression vectors which comprise the 5′ UTR of the mRNA variant to be investigated at the 5′ end of a marker gene are transfected into cultivated cells, tissue samples or organisms. If the intention is to investigate the translation efficiency of reporter gene-mRNAs having different 5′ UTRs as a function of various cell types, tissues or organisms, the reporter gene constructs are designed as follows. The 5′ UTR of an mRNA to be investigated is ligated between the 3′ end of a viral promoter (CMV, RSV or SV40 promoter) and the 5′ end of the coding region of a reporter gene (photinas pyralis luciferase, renilla reniformis luciferase, chloramphenicol transferase (CAT), β-galactosidase, GFP or others). This expression construct is expressed in cultivated cells, tissue samples or organisms. In order to compensate for variations in the translation efficiency, a further reporter gene construct is cotransfected. The dual luciferase system (Promega) is suitable for this, because both the actual measurement (photinas pyralis luciferase) and the expression of the control construct (renilla reniformis luciferase) can be carried out with this system in one mixture [71, 72]. The luciferase activity in the various mixtures is determined in a luminometer (Luciferase Assay, Promega, [26]. The baseline (100%) used for all measurements is the luciferase activity in mixtures which comprise lysates of cells, tissues or organisms transfected with a reporter gene vector which codes for a photinas pyralis luciferase mRNA whose 5′ UTR comprises exclusively a Kozak consensus sequence [7, 8]. The influence of cellular factors which are expressed in a particular cell type, tissue or organism on the translation of an mRNA to be investigated is determined by comparing the translation efficiency of one or more mRNAs to be investigated in vitro and in vivo. Factors which influence the CAP-dependent and CAP-independent translation of various mRNAs include translation initiation factors [60, 61], tumor suppressors such as p53 [62, 63] and a number of other proteins [64, 65].

The joint influence of the 5′ UTR and of the coding region on the translation efficiency of an mRNA to be investigated cannot be determined by reporter gene assays in which the expression rate is measured by means of the enzymatic activity of a reporter protein. The folding of a fusion protein whose amino-terminal half consists of a protein to be investigated and whose carboxy-terminal half consists of a reporter protein is often different from that of the two unfused proteins. The enzymatic activity of the reporter protein portion in fusion proteins therefore depends on the protein to which the reporter protein is fused. In order to circumvent this problem, the protein to be investigated is fused at the carboxy terminus to a short marker peptide. This marker peptide may be inter alia a CBP tag (calmodulin-binding peptide; Stratagene), FLAG tag (Sigma-Aldrich) or a His tag (5-7 consecutive histidine residues) [73, 74]. The mRNA variants which are to be investigated and which are transcribed from one or more genes are amplified with the aid of RT-PCR [26] and isolated. The 5′ end of the 5′ primers used corresponds to the 5′ end of the various 5′ UTRs, and the 3′ primers used correspond to the 3′ end, i.e. to the last codon in the coding region of the mRNA to be investigated (the stop codon is omitted). The PCR products are ligated into an expression plasmid between the 3′ end of a viral promoter (CMV, RSV, SV40 and others) and the 5′ end of the sequence coding for the marker peptide, so that the coding region of the mRNA to be investigated is fused to the sequence coding for the marker peptide. The plasmid vectors for expressing the fusion proteins described above are commercially available (Qiagen, Clontech, Stratagene). Transfection of E. coli host strains with the plasmids, replication of the plasmids, and isolation of the plasmid DNA takes place in accordance with standard biotechnology laboratory protocols [26]. Various cell types, tissues or organisms to be investigated are transfected with the expression constructs described above, which comprise the cDNA sequence of the 5′ UTR and of the coding region of the various mRNA variants transcribed from one or more genes. To determine the transfection efficiency, a reporter gene plasmid which expresses photinas pyralis luciferase or renilla reniformis luciferase is cotransfected. The translation efficiency of the various mRNA variants expressed by expression plasmids is determined by Western blotting or slot blotting methods [65, 66].

The fusion proteins are detected with the aid of an antibody or protein which binds the marker peptides specifically. Quantitative detection of proteins takes place by standard biotechnology laboratory protocols. The baseline value (100%) used for all measurements is the detectable amount of fusion protein in mixtures comprising lysates of cells, tissues or organisms which have been transfected with an expression construct which harbors the cDNA sequence of an mRNA variant to be investigated, whose 5′ UTR comprises exclusively a Kozak consensus sequence [7, 8]. Besides the influence of the 5′ UTR and cellular factors on the translation of an mRNA to be investigated, additionally the influence of the sequence of the coding region on the translation of the mRNA variant to be investigated is determined by comparing the translation efficiency of reporter gene-mRNAs which have the 5′ UTR of mRNA variants to be investigated which are transcribed from one or more genes, with the translation efficiency of the complete mRNA variants. The same expression constructs as described above are used to determine under various external influences the translation efficiency of the mRNA variants to be investigated. Detection of the translation efficiency of the mRNA variants to be investigated takes place by measuring the enzymatic activity of a reporter gene or immunochemical detection of a protein fused to a marker peptide (see above). The cells transfected with expression plasmids are exposed to various external influences which may include inter alia a change in the oxygen partial pressure, in the nutrient supply, in the temperature, in the atmospheric pressure, and the effect of cytokines, hormones, cytostatics or other drugs. The measurements described here are carried out in the cell lines which are present in the NCI-60 panel [35] and which have been very comprehensively characterized. In addition, the translation efficiency of mRNA variants to be investigated is determined in clinical samples and other established cell lines.

Measurement of the Effect of External Influences on Cellular Functions such as Growth, Apoptosis or Proliferation

The effect of external influences on cells, tissues or organisms to be investigated is determined on the basis of a number of parameters which may include inter alia the apoptosis rate, the proliferation rate and cell growth. The external influences mentioned herein may include inter alia a change in the oxygen partial pressure, in the nutrient supply, in the temperature, in the atmospheric pressure, and the effect of cytokines, hormones, cytostatics or other drugs. Cells, tissues or organisms to be investigated are maintained in culture and exposed to one or more defined external influences for 24 h -48 h. To determine the effect of different dose levels of the external influence to be investigated, inter alia cell growth, apoptosis rate and/or proliferation rate are determined in the treated cells. The determination of the growth rate, proliferation rate and/or apoptosis rate in cultured cells takes place in accordance with standard biotechnology or cell biology laboratory protocols [75, 76, 77, 78 and 79]. The amount of an external influence which, for example, inhibits cell growth by 50% (GI₅₀→Growth Inhibition) [35] is ascertained by extrapolating the growth rate, apoptosis rate or proliferation rate with different dose levels of one or more external influences on one or more cell types, tissues or organisms. Based on the apoptosis rate or the proliferation, the dose of an external influence in which apoptosis is induced in 50% of the investigated cells (AI₅₀→Apoptosis Induction) or proliferation is inhibited by 50% (PI₅₀→Proliferation Inhibition) is ascertained.

Integration of Clinical Data

If the system is to be employed for diagnosis of neoplastic diseases, the database module of component 2 may include data on the type of therapy which the drug employed, the dosage of the drugs, the tolerability or effect of the drugs employed for the therapy, the time between the initial disorder and the appearance of recurrences or metastases, and one or more expression profiles, produced with component 1 of the system, of the investigated tumors. If pathological states such as neurodegenerative syndromes, autoimmune diseases, cardiovascular disorders, viral infections or drug resistances are to be analyzed, the database module of component 2 will preferably comprise data on the type of therapy which the drug employed, the dosage of the drugs, the tolerability or effect of the drugs employed, and one or more expression profiles, produced with component 1 of the system, of diagnostically relevant tissue samples.

Analysis & Interpretation

Analysis and interpretation of the expression data produced with component 1 (DNA array) is carried out at two levels with the aid of the database and analysis module present in component 2. At the first level of interpretation, a translation efficiency is assigned to every mRNA variant identified and quantified with the aid of component 1. At the second level of interpretation, the complete expression profile produced with component 1 is compared with other expression profiles present in the database of component 2, and assigned to a particular expression type. This assignment to a particular expression type makes it possible to determine the translation efficiency of all mRNA variants identified and quantified at level 1 of interpretation as a function of cellular factors, and to identify the mRNA preferentially translated in the investigated cell type, tissue or organism.

Prediction of the Protein Concentration

The measurements required to predict the amount of one or more proteins present in a cell type, tissue or organism to be investigated include the total transcription rate of the mRNA variants coding for one or more particular proteins, and the transcription rate of the individual mRNA variants coding for these proteins, and are determined with component 1 of the system (DNA array). The transcription rate of one or more particular mRNAs is determined in component 1 (DNA array) on the basis of the intensity of the hybridization signals specific for the mRNA to be investigated. To standardize the hybridization signals of the mRNA variants to be investigated with the corresponding probe nucleic acids in component 1, the intensity of the hybridization signals from mRNAs which are transcribed in all cell types, tissues or organisms (called housekeeping genes) is measured. Expression of the housekeeping genes employed for standardization of the hybridization signals cannot be checked at the level of translation. A data record which comprises the translation efficiency of this mRNA variant compared with mRNA variants transcribed from the same and/or other genes, the dependence of the translation efficiency on cellular factors, and the mRNA variant preferentially translated in a particular cell type, tissue or organism, is assigned to each probe nucleic acid on component 1 (DNA array) and each group of probe nucleic acids which represents a particular mRNA variant. Comparison of an expression profile produced by a tissue sample with the expression profiles present in the database module of component 2 makes it possible to assign the investigated sample to a particular cell or tissue type and thus to assess the translational state ,of the investigated cell type or tissue. The product of the cell type- or tissue-specific translation efficiency (P_{(RNA-Var.lx)}) of one or more mRNA variants to be investigated, and the transcription rate (T_{(RNA-Var.lx)}), measured with the aid of component 1, of the mRNA variants to be investigated gives a value (C_{prot.x}) which corresponds to the amount present in the investigated tissue of the protein(s) corresponding to the mRNA variants. The following therefore applies:
(I_{(RNA-Var.1x)})/(I_{(Housekeeping)})=T_{(RNA-Var.1x)}
T_{(RNA-Var.1x)}×P_{(RNA-Var.1x)}=C_{Prot.x}
References
[0] Levin, B.: “Genes VI” 1997 Oxford University press
[1] Willis, A. E.: “Translational control of growth factor and proto-oncogene expression”, 1999, Int. J. Biochem. Cell Biol., vol. 31
[2] Harigan, M. et al.: “A cis-acting element in the bcl-2 gene controls expression through translational mechanisms”, 1996 Oncogene, vol. 12
[3] Jagus, R. et al.: “PKR, apoptosis and cancer”, 1999, Int. J. Biochem. Cell Biol., vol. 31
[4] Ewen, M. E. & Miller, S. J.: “p53 and translational control”, 1996, Biochim. Biophys. Acta, vol. 1242
[5] Landers, J. E. et al.: “Translational enhancement of mdm2 oncogene expression in human tumor cells containing a stabilized wild-type p53 protein”, 1997, Cancer Res. vol. 57
[6] Clemens, M. J. & Bomer, A. U.: “Translational control: The cancer connection”, 1999, Int. J. Biochem. Cell Biol., vol. 31
[7] Kozak, M.: “An analysis of 5′-noncoding sequences from 699 vertebrate messenger RNAs”, 1987, Nuc. Acids Res. vol. 15
[8] Kozak, M. “An analysis of vertebrate mRNA sequences: intimations of translational control”, 1991, J. Cell Biol., vol. 115
[9] El-Deiry, W. S.; “Regulation of p53 downstream genes”, 1998, Seminars in CANCER BIOLOGY, vol. 8
[10] Kozak, M.: “Adherence to the first-AUG rule when a second AUG codon follows closely upon the first”, 1995, Proc. Natl. Acad. Sci. U.S.A., vol. 92
[11] van der Velden, A. W. & Thomas, A. A. M.: “The role of the 5′ untranslated region of an mRNA in translation regulation during development”, 1999, Int. J. Biochem. Cell. Biol., vol. 31
[12] Tsujimoto, Y. & Croce C. M.: “Analysis of the structure, transcripts, and protein products of bcl-2, the gene involved in human follicular lymphoma”, 1986, Proc. Natl. Acad. Sci. U.S.A., vol. 83
[13] Seto, M. et al.: “Alternative promoters and exons, somatic mutation and deregulation of the Bcl-2-lg fusion gene in lymphoma”, 1988, EMBO J., vol. 7
[14] Kamoshita, N. et al.: “Genetic analysis of internal ribosome entry site on Hepatitis C virus RNA: Implication for involvement of the highly ordered structure and cell type-specific transacting factors”. 1997, Virology, vol. 233
[15] Jang, S. K. et al.: “Cap-independent translation of encephalomyocarditis virus RNA: structural elements of the internal ribosome entry site and involvement of a cellular 57-kD RNA-binding protein”, 1990, Genes Dev., vol. 4
[16] Soo-Kyung, O. H. et al.: “Homeotic gene Antennapedia mRNA contains 5′-noncoding sequences that confer translational initiation by internal ribosome binding”, 1992, Genes Dev., vol. 6
[17] Huez, I. et al.: “Two independent internal ribosome entry sites are involved in translation initiation of vascular endothelial growth factor mRNA”, 1998, Mol. Cell. Biol., vol. 18: 11
[18] Vagner, S. et al.: “Alternative translation of human Fibroblast Growth Factor 2 mRNA occurs by internal entry of ribosomes”, 1995, Mol. Cell Biol., vol. 15; 1
[19] Macejak, D. G. & Sarnow, P.: “Internal initiation of translation mediated by the 5′ leader of a cellular mRNA”, 1991, Nature, vol. 353
[20] Yang, Q. & Samow, P.: “Location of the internal ribosomne entry site in the 5′ non-coding region of the immunoglobulin heavy-chain binding protehi (BiP) mRNA: evidence for specific RNA-protein interactions”, 1997, Nuc. Acids Res., vol. 25; 14
[21]Bernstein, J. et al.: “PDGF2/c-sis mRNA leader contains a differentiation linked internal ribosome entry site (D-IRES)”, 1997, J. Biol. Chem., vol. 272; 14
[22] Gan, W. & Rhoads, R. E.: “Internal initiation of translation directed by the 5′-untranslated region of the mRNA for eIF4G, a Factor involved in the Picomavirus-induced switch from Cap-dependent to internal initiation”, 1996, J. Biol. Chem., vol. 271: 2
[23] Nanbru, C. et al.: “Alternative translation of proto-oncogene c-myc by an internal ribosome entry site”, 1997, J. Biol. Chem., vol. 272; 51
[24] M. Zuker, M. et al.; “Algorithms and Thermodynamics for RNA Secondary Structure Prediction: A Practical Guide”, In RNA Biochemistry and Biotechnology, 11-43, J. Barciszewskl & B. F. C. Clark, eds., NATO ASI Series, Kluwer Academic Publishers, Dordrecht, NL, (1999)
[25] Links, M. & Brown, R.: “Clinical relevance of the molecular mechanisms of resistance to anti-cancer drugs”, 1999, Expert Reviews in Molecular Medicine, ISSN 1462-3994
[26] Sambrook, J. et al.; “Molecular Cloning” 2001, 3^{rd} Edition, Cold Spring Harbor Laboratory
[27] Nielsen, P. E. et al.: “Peptide nucleic acids: Protocols and Applications”, 1999, Horizon Scientific Press
[28] Kumar, R. et al.: “Nuclease protection assays”, U.S Pat. No. 5,770,370; WO 97/47640
[29] Pradet-Balade, B. et al.: “Translation control: bridging the gap between genomics and proteomics?”, 2001, TIBS, vol. 26; 4
[30]Celis, J. E. et al.: “Gene expression profiling: monitoring transcription and translation products using DNA microarrays and proteomics”, 2000, FEBS Lett., vol. 480
[31] Hentze, M. W.:“Improved predictive power of RNA analysis for protein expression”, WO 00/68423
[32] Einat, P. et al.:“Method for identifying translationally regulated genes”. U.S. Pat. No. 6,013,437; WO 98/21321
[33] Einat, P. et al.:“Method for identifying genes”, WO 99/58718
[34] Martinez-Salas, E. et al.: “Functional interactions in internal translation initiation directed by viral and cellular IRES elements”, 2001, J. Gen. Virol., vol. 82
[35] Scherf, U. et al.: “A gene expression database for the molecular pharmacology of cancer”, 2000, Nature genetics, vol. 24
[36] Qiagen: RNeasy Midi/Maxi Handbook 06/2001
[37] Wallace. R. B. et al.: “Hybridization of synthetic oligodeoxyribonucleotides to phi chi 174 DNA: the effect of single base pair mismatch”, 1979, Nuc. Ac. Res., Vol. 6
[38] Howley, P. M. et al.: “A rapid method for detecting and mapping homology between heterologous DNAs. Evaluation of polyomavirus genomes”, 1979, J. Biol. Chem., Vol. 254
[39] Breslauer, K. J. et al.: “Predicting DNA duplex stability from the base sequence”, 1986, Proc. Natl. Acad. Sci., Vol. 83
[40] Freler, S. M. et al.: “Improved free-energy parameters for predictions of RNA duplex stability”, 1986, Proc. Natl. Acad. Sci., Vol. 83
[41] Sugimoto, N. et al.: “Improved thermodynamic parameters and helix initiation factor to presict stability of DNA duplexes”, 1996, Nuc. Ac. Res., Vol. 24, No. 22
[42] SantaLucia jr., J. et al.: “Improved nearest neighbor parameters for predicting DNA duplex stability”, 1996, J. Biol. Chem., Vol. 35
[43] Qiagen and others, polyA+mRNA isolation
[44] Boehringer Mannheim: RNAse Protection kits
[45] Steemers, F. J. et al.: “Screening unlabeled DNA targets with randomly ordered fiber-optic gene arrays”, 2000, Nature Biotech., Vol. 18
[46] Fodor, S. P. A. et al.: “Light-directed, spatially addressable parallel chemical synthesis”, 1991, Science, Vol. 251
[47] Lipshutz, R. J. et al., “High density synthetic oligonucleotide arrays”, 1998, Nature Genet., Vol. 21
[48] Blanchard, A. P. et al.: “High density oligonucleotide arrays”, 1996, Biosensors & Bioelectronics, Vol. 11
[49] Fodor, S. P. A. et al. U.S. Pat. No. 5,424,186:
[50] Schena, M.: “DNA-Microarrays: Apractical approach”, 1999, Oxford University Press
[51] Schena, M, et al.: Parallel human genome analysis: Microarray-based expression monitoring of 1000 genes”, 1996, Proc, Natl. Acad. Sci., Vol. 93
[52] Gaitt, M. J., “Oligonucleotide-synthesis: A practical approach”, 1984, Oxford University Press
[53] Kricka, L.; “Non isotopic DNA probe techniques”, 1992, Academic Press, San Diego
[54] “Fluorescent and Luminescent Probes for biological activity”, 1999, 2^{nd} Edition, Mason, W. T. ed.
[55] Worley, J. M. et al., 1994, Molecular Dynamics Application Note #57
[56] Anderson, M. L. M.:“Nucleic acid Hybridization”, 1998, Springer-Verlag Telos
[57] Bowtell, D. D. L.: “Options available-from start to finish for obtaining expression data by microarray”, 1999, Nature Genet., Vol. 21
[58] Gelfand, D. H. et al.:“Detection of specific polymerase chain reaction product by utilizing the 5′ to 3′ exonuclease activity of Thermus aquaticus DNA-polymerase”, 1991, Proc. Natl. Acad. Sci., Vol. 88 and U.S. Pat. No. 5,210,015 (1993)
[59] Tyagi, S. et al.:“Molecular Beacons: probes that fluoresce upon hybridization”, 1996, Nature Biotech., Vol. 14
[60] Hayashi, S. et al.:“Increase in Cap- and IRES-Dependent Protein Synthesis by Overproduction of Translation Initiation Factor eIF4G”, 2000, Biochem. Biophys. Res. Com., Vol. 277
[61] Gingras, A. -C., et al.:“eIF4 Initiation Factors: Effectors of mRNA recruitment to ribosomes and regulators of translation”, 1999, Annu. Rev. Biochem., Vol. 68
[62] Miller, S. J., et al.:“p53 Binds Selectively to the 59 Untranslated Region of cdk4, an RNA Element Necessary and Sufficient for Transforming Growth Factor b- and p53- Mediated Translational Inhibition of cdk4”, 2000, Mol. Cell. Biol., Vol. 20, No. 22
[63] Ewen, M. E. et al.;“p53 and translational control”, 1996, Biochim. Biophys. Acta, Vol. 1242
[64] Holcik, M. et al.;“lnternal ribosome initiation of translation and the control of cell death” 2000, Trends Genet., Vol 16, No. 10
[65] Laemmil, U. K.:“Cleavage of structural proteins during the assembly of the head of bacteriophage T4”, 1975, Nature, Vol. 227
[66] Towbin, H.: et al.:“Electrophoretic transfer of proteins from polyacrylamide gels to nitrocellulose sheets: Procedure and somer applications”, 1979, Proc. Natl. Acad. Sci., Vol. 76
[67] Harlow, E. et al.: “Antibodies: A Laboratory Manual”, 1988, Cold Spring Harbor Laboratory Press
[68] deWet, J. R. et al.: “Cloning of firefly luciferase cDNA and the expression of active luciferase in Escherichia coli”, 1985, Proc. Natl. Acad. Sci., Vol. 82
[69] Alam, J. et al.: “Reporter genes: application to the study of mammalian gene transcription”, 1990, Anal. Biochem., Vol. 188
[70] Wood, K. V.: “Firefly luciferase: a new tool for the molecular biologists”, 1990, Promega Notes 28, 1
[71] Farr, a. et al.: “A pitfall of using a second plasmid to determine transfection efficiency”, 1991, Nuc. Acids, Res., Vol. 20
[72] Sherf, B. A. et al.: “Dual-Luciferase® reporter-assay: an advanced co-reporter technology intergrafing firefly and Renilla luciferase assays”, 1996, Promega Notes 57, 2
[73] Janknecht, R. et al.: “Rapid and efficient purification of native histidine-tagged protein expressed by recombinant vaccinia virus”, 1991, Proc. Natl. Acad. Sci., Vol. 88
[74] Pogge von Strandmann, E. et al.: “Highly specific and sensitive detection of 6xHis tagged proteins using MRGS His Antibody”, 1996, QIAGEN News, No. 1, 9
[75] Spector, D. L. et al.: “Cells: A Laboratory Manual”, 1998, Cold Spring Harbor Laboratory Press
[76] Van Furth, R. et al.: “Immuno-cytochemical detection of 5-bromo-2-deoxyuridine incorporation in individual cells”, 1988, J. Immunol. Methods, Vol. 108
[77] Gold, R. et al.: “Differentiation between cellular apoptosis and necrosis by the combined use of in situ tailing and nick translation techniques”, 1994, Lab. Invest., Vol. 71
[78] Vermes, I. et al.: “A novel assay for apoptosis, Flow cytometric detection of phosphatidylserine expression on early apoptotic cells using fluorescein labelled Annexin V”, 1995, J. Immunol. Methods, Vol. 184
[79] Scudiero, E. A. et al.: “Evaluation of a soluble tetrazolium/formazan assay for cell growth and drug sensitivity in culture using human and other tumor cell lines”, 1988, Cancer Res., Vol. 48
[80] Cory, A. H. et al.: “Use of an aqueous soluble tetrazolium/formazan assay for cell growth assays in culture”, 1991, Cancer Commun., Vol. 3

1-31. (Cancelled).

32. A substrate for use in transcription analysis, comprising:
(a) a solid matrix; and
(b) at least first and second polynucleotide probes immobilized to a surface of the solid matrix, the probes being complementary to at least a portion of a genomic polynucleotide sequence for a gene, each probe comprising from about ten to about forty nucleotides, wherein
(i) the first probe is complementary to at least a portion of a first mRNA variant from the genomic polynucleotide or a portion of a cDNA corresponding to the first mRNA variant,
(ii) the second probe is complementary to at least a portion of the first mRNA variant or at least a portion of a cDNA corresponding to the first mRNA variant and is also complementary to at least a portion of a second mRNA variant from the genomic polynucleotide or at least a portion of a cDNA corresponding to the second mRNA variant, and
(iii) the first probe is not complementary to the second mRNA variant or to a CDNA corresponding to the second mRNA variant.

33. The substrate according to claim 32, further comprising at least a third polynucleotide probe immobilized to a surface of the solid matrix and comprising from about ten to about forty nucleotides, wherein:
(a) the third probe is complementary to at least a portion of the first and second mRNA variants or at least a portion of the cDNA corresponding to the first and second mRNA variants and is also complementary to at least a portion of a third mRNA variant from the genomic polynucleotide or at least a portion of a cDNA corresponding to the third mRNA variant; and
(b) the first and second probes are not complementary to the third mRNA variant or to a cDNA corresponding to the third mRNA variant.

34. The substrate according to claim 32, wherein at least one of the probes immobilized on the surface of the solid matrix comprises a polynucleotide sequence which is complimentary to at least a portion of the coding region of the gene.

35. The substrate according to claim 32, wherein the probes immobilized on the surface of the solid matrix comprise substantially the complete genomic nucleotide sequence of the 5′ or 3′ noncoding region of the gene.

36. The substrate according to claim 32, wherein the probes immobilized on the surface of the solid matrix comprise substantially the complete genomic nucleotide sequence of the noncoding region of the gene.

37. The substrate according to claim 32, wherein the probes immobilized on the surface of the solid matrix comprise substantially the complete genomic nucleotide sequence of the gene.

38. The substrate according to claim 32, further comprising at least one additional probe, wherein each additional probe comprises from 10 to 40 nucleotides, and each additional probe is complementary to at least a portion of the nucleotide sequence of a gene selected from the group consisting of housekeeping genes of the organism from which the gene to be analyzed originates, bacterial genes, plant genes and combinations thereof.

39. The substrate according to claim 32, wherein the solid matrix is a DNA array.

40. A method for analyzing transcription and translation, comprising:
(a) identifying each mRNA variant encoding a polypeptide present in a sample;
(b) quantifying the amount of each mRNA variant identified in step (a);
(c) determining the respective translation efficiency of each mRNA variant identified in step (a); and
(d) calculating the amount of the polypeptide present in the sample based on the results of steps (b) and (c).

41. A method for analyzing transcription and translation, comprising:
(a) preparing a plurality of mRNA variants or derivatives thereof from a sample;
(b) contacting the mRNA variants or derivatives thereof with a substrate comprising:
(i) a solid matrix; and
(ii) at least first and second polynucleotide probes immobilized to a surface of the solid matrix, the probes being complementary to at least a portion of a genomic polynucleotide sequence for a gene, each probe comprising from about ten to about forty nucleotides, wherein:
(1) the first probe is complementary to at least a portion of a first mRNA variant obtained from the sample or a portion of a cDNA corresponding to the first mRNA;
(2) the second probe is complementary to at least a portion of the first mRNA variant obtained from the sample or at least a portion of a cDNA corresponding to the first mRNA variant and also is complementary to at least a portion of a second mRNA variant obtained from the sample or at least a portion of a cDNA corresponding to the second mRNA variant; and
(3) the first probe is not complementary to the second mRNA variant or to a cDNA corresponding to the second mRNA variant;
(c) identifying each mRNA variant or derivative thereof from step (a) that binds to the probes;
(d) quantifying the amount of each mRNA variant or derivative thereof identified in step (c);
(e) determining the respective translation efficiency of each mRNA variant or derivative thereof identified in step (c); and
(f) calculating the amount of the polypeptide present in the sample based on the results of steps (d) and (e).

42. The method according to claim 41, wherein the sample originates from a culture of mammalian cells, a tissue or an organ of a mammal.

43. The method according to claim 41, wherein the mRNA variants or derivatives thereof are selected from the group consisting of total RNA, polyA+ RNA, cRNA, cDNA and combinations thereof.

44. The method according to claim 41, wherein the mRNA variants or derivatives thereof are labeled before carrying out step (b).

45. The method according to claim 41, wherein at least two different mRNA variants or derivatives thereof are transcribed from the gene to be analyzed.

46. The method according to claim 45, wherein the mRNA variants or derivatives thereof differ at the 5′ end, differ at the 3′ end and/or represent different splice forms of the gene.

47. The method according to claim 41, wherein step (f) is carried out by a database module and an analysis module.

48. The method according to claim 47, wherein the database module comprises a storage medium on which the respective translation efficiencies of the mRNA variants or derivatives thereof are stored.

49. The method according to claim 47, wherein the analysis module comprises a processor and a storage medium.

50. A kit for analyzing the expression of at least one gene in a sample, comprising:
(a) as a first component, a solid matrix as claimed in claim 32;
(b) as a second component, a storage medium on which the respective translation efficiencies of the mRNA variants or derivatives thereof are stored.

51. The kit according to claim 50, further comprising a device for determining the respective amounts of the mRNA variants or derivatives thereof, which are bound to the respective probes after contacting the mRNA variants or derivatives thereof with the substrate.

52. The kit according to claim 50, wherein the second component further comprises a transcription profile derived from cells, tissues, or organisms from which the sample is derived.

53. The kit according to claim 50, wherein the second component further comprises a transcription profile derived from cells, tissues or organisms altered by a disease.

54. The kit according to claim 53, wherein the disease is selected from the group consisting of neurodegenerative disorders, cancer, autoimmune diseases, chronic disorders of the elderly, cardiovascular disorders, viral diseases and drug resistances.

55. The kit according to claim 50, wherein the second component further comprises a transcription profile derived from tumor cells which have been treated with one or more therapeutic agents.

56. A method for determining or analyzing disorders, comprising comparing a transcription profile produced by means of the substrate as claimed in claim 32 with transcription profiles of pathologically altered cells, tissues or organisms.

57. A method for determining or analyzing the effects of external influences on a sample, comprising comparing a transcription profile produced by the substrate as claimed in claim 32 to a transcription profile of the same sample after exposure to an external influence.

58. A method for determining the secondary structure of an RNA, comprising:
(a) partially digesting an RNA with an RNAse; and
(b) contacting the RNA digest from step (a) with the substrate claimed in claim 32.
